(19) United States
(12) Patent Application Publication
(10) Pub. No.: US 2024/0001196 A1
(43) Pub. Date: Jan. 4, 2024

(54) Artificial Intelligence Assisted Personal Training System, Personal Training Device and Control Device

(71) Applicant: THE HONG KONG POLYTECHNIC UNIVERSITY, Hong Kong (CN)

(72) Inventors: Yiu-Wan Joanne YIP, Hong Kong (CN); Ka-Wing Frances WAN, Hong Kong (CN); Ting Hin Alex MAK, Hong Kong (CN); Wai Sze CHAN, Hong Kong (CN); Wing Ki CHAN, Hong Kong (CN); Hiu Ching LUI, Hong Kong (CN); Yeok-Tatt CHEAH, Hong Kong (CN)

(21) Appl. No.: 18/188,560

(22) Filed: Mar. 23, 2023

(30) Foreign Application Priority Data
Jul. 4, 2022 (CN) 202210781291.7

(51) Int. Cl. A63B 24/00; A63B 71/06; G06N 3/045

(52) U.S. Cl. CPC A63B 24/0062; A63B 24/0075; A63B 71/0622; G06N 3/045; A63B 2220/44; A63B 2220/836; A63B 2230/08; A63B 2230/62

(57) ABSTRACT

A personal training system assisted by artificial intelligence (AI) has a personal training device and a control device. The personal training device includes a jacket and pants, and houses sensors at positions corresponding to a user's main muscle groups for monitoring the user's movement posture and muscle activity of the main muscle groups. The control device has a data preprocessing unit for processing detected-signal data of the sensors, a training analysis device for executing an AI algorithm to conduct fatigue analysis of the user's movement based on the user profile and the detected-signal data, and to make training load recommendations. The personal training system can simultaneously monitor posture, muscle activity and muscle fatigue in real time during the exercise; and use the AI algorithm to evaluate exercise performance and provide real-time feedbacks to improve the exercise and training efficiency, and reduce the risk of injury.

### CROSS-REFERENCE TO RELATED APPLICATIONS

This application claims priority to, and the benefit of, China Patent Application No. 202210781291.7 filed on Jul. 4, 2022, the disclosure of which is hereby incorporated by reference in its entirety.

### TECHNICAL FIELD

The present invention relates to an artificial intelligence (AI) assisted personal training system, a personal training device and a control device.

### BACKGROUND

The benefits of exercise are considered to “solve one of the biggest public health problems of the 21st century” and are receiving increasing public attention worldwide. Exercise is Medicine® is a global health initiative administered by the American College of Sports Medicine (ACSM) that recognizes the importance of physical activity for optimal health and encourages physicians and other healthcare providers to provide patients with physical activities when designing the patients' treatment plans. In fact, more and more people are trying to incorporate exercise into their daily lives and develop the habit of regularly doing exercise. According to certain reports, between 2009 and 2017, the number of gymnasiums in Hong Kong increased from 548 to 743 (a growth rate of about 35.6%). Resistance training (also known as strength training or weight training) has rapidly become an emerging fitness trend in recent years. Resistance training is a form of physical activity designed to increase strength, anaerobic endurance and skeletal muscle size through muscle contraction against resistance.

However, sports may lead to injuries. The main reasons may include: insufficient training level, poor physical fitness, incorrect movement, lack of self-protection ability, no preparatory activity or insufficient preparatory activity before doing exercise, lack of training to adapt to the environment, and improper organization of teaching and competition works. In 2018, sports and equipment-related injuries ranked the second among all injury types in the U.S. for 25- to 44-year-olds. Proper posture and use of muscles during athletic activities not only maximizes training efficiency, but also minimizes additional stress on ligaments and joints, thereby reducing the risk of injury.

There are already smart clothing or wearables on the market that can monitor heart rate, posture, body temperature, stress or stress levels when the user is doing exercise. Table 1 and FIGS. 15A-15C show a comparison of different aspects of current smart clothing technology with embedded sensors for monitoring biometrics. The latest surface electromyography (sEMG) and sports wearable sensor technologies allow for simultaneous and accurate measurement of muscle activity, body posture and body movement. However, the wearable technology on the market today does not meet the demands of more advanced physical exercises such as resistance training.

TABLE 1
Comparison of current smart clothing technologies.

| | | | Nadi X Yoga |
|---|---|---|---|
| Product Name | Athos Training System | Hexoskin | Pants |
| Company | Mad Apparel company, | Cane Technology | Wearable X |
| | USA | company, | company, |
| | | Montreal, Canada | Sydney, Australia |
| Features | Monitor muscle activity, | Monitors 1-lead ECG, | Monitors yoga |
| | heart rate, breathing rate, | heart rate, heart rate | poses, provides |
| | calorie burn, and active | variability, heart rate | guided vibrations |
| | versus rest periods | recovery, respiratory | for various yoga |
| | | rate, minute ventilation, | poses, provides |
| | | activity level, calories | voice guidance via |
| | | burned, peak | mobile phone |
| | | acceleration, steps, | |
| | | cadence, position, speed | |
| | | and sleep | |
| Communication | Sensors embedded in the | Collected data is | Pants with |
| capabilities | garments read biosignals | synchronized with | integrated sensor |
| | and transmit the data via | mobile app (iOS and | function and |
| | Bluetooth to a mobile | Android compatible) | controls ″Pulse” |
| | app (compatible with | via Bluetooth and with | clipped to the |
| | iOS only) showing | PC via HxDashboard | pants behind the |
| | which muscles are firing | software for raw and | left knee to |
| | and how much they are | processed data | provide haptic |
| | moving. | visualization | feedback |
| | | | (vibration) |
| Price | Men's shirt (with 1 | Professional Kit | Yoga Pants + |
| | core): US$398; | (Hexoskin Professional | “Pulse”: US$249 |
| | Shorts/Women's | Shirt + Smart Device): | |
| | Leggings (with 1 core): | US$579. | |
| | US$348; | | |
| | Shirt + shorts (with 2 | | |
| | cores): US$696. | | |

The prior-art products described above are shown in FIGS. 15A-15C, with further details as elaborated as follows.

The Athos training system from Mad Appareal in the United States includes smart clothing that can be adapted to any type of training, such as T-shirts, shorts, and tights with embedded sEMG. This training system can provide feedbacks to users through a mobile app that is only compatible with iOS, and provide reports after training sessions as well as weekly reports. However, it requires the coach/athlete to read the report or weekly report, to manually assess the wearer's or user's performance and progress or to provide real-time performance feedback.

Hexoskin from Cane Technologies of Montreal, Canada is a smart garment that can be used to continuously monitor heart, lung, activity and sleep data. It includes smart sports vests, jerseys and body condition sensors. It is equipped with sensors to monitor heartbeat, breathing and body condition, and is suitable for data tracking during exercise and during sleep, helping users understand their breathing rates, heart rates, sweating behavior, etc. It can also calculate the number of footsteps taken every day, the amount of calories burned, etc. The device has a companion mobile app (APP), but is also compatible with most popular third-party health apps. It still requires professionals (such as fitness trainers) to interpret the collected data to provide feedbacks on the user's exercise performance, exercise intensity, and exercise posture.

The Nadi X yoga pants from Wearable X in Sydney, Australia are smart clothing that can monitor movement posture and can be connected to a mobile phone to provide voice guidance. Nadi X yoga pants have sensors on the lower back, knees, feet, etc., and are powered by a battery above the left knee to accurately collect user data. Nadi X yoga pants can be connected to the exclusive App via Bluetooth, and after the connection, the user's training progress can be tracked. Users can choose the yoga difficulty that suits them to start training. Nadi X yoga pants collect the user's posture through the above sensors. When the user's yoga posture is not standard, the sensor will vibrate to different degrees, and the hips, knees and ankles will vibrate softly. There are ways to improve posture and remind users to adjust to the correct movement, thus helping users to adopt more correct yoga movements and effectively achieve movements that may not have been possible in the past.

Despite availability of the above-mentioned prior-art products, current smart wearable technologies cannot satisfy more advanced physical exercises, such as insufficient performance and efficiency evaluation of advanced physical exercises, and cannot provide real-time feedbacks and suggestions through AI algorithms. The latest sEMG and sports wearable sensor technologies allow for simultaneous and accurate measurement of muscle activity, body posture and body movement. The most common smart wearable devices on the market, such as compression T-shirts, leggings, etc., are capable of measuring the user's body temperature, heart rate, heart rate variability, respiratory rate, calorie consumption, steps, and more. However, the biosignals provided by current wearable devices are insufficient to assess the performance and efficiency of advanced physical exercises, such as resistance exercise tasks. To the Inventors' knowledge, the Athos training system described in Table 1 is the only wearable technology on the market that can measure the activity of major muscle groups. However, its functionality is still premised on the biometric tracking phase.

Second, people who are physically active often consult personal fitness trainers to pursue more advanced training goals, such as resistance training. Professional fitness trainers have an extensive knowledge of muscle anatomy and a good understanding of the exact muscle groups that need to be activated and deactivated during different movement tasks. Most importantly, a qualified fitness trainer can provide immediate feedback on whether certain exercises/tasks have been performed correctly and provide guidance to correct mistakes, which is essential for building muscle. However, existing wearables are unable to provide precise real-time feedbacks and recommendations through AI algorithms.

The Inventors have found that with the help of the latest technologies in wearable sensors and AI methods, with proper data input and training, the knowledge and skills of fitness training trainers are likely to be replaced by AI. The present invention has been developed based on this observation.

### SUMMARY

A first aspect of the present invention is to provide a personal training device.

The training device comprises an upper garment part, a trousers part and plural sensor accommodating units. The upper garment part and trousers part are both used for wearing by a user. The sensor accommodating units are distributed on the upper garment part and trousers part. Plural sensors are installed in the sensor accommodating units such that an individual sensor accommodating unit is equipped with one or more of the sensors. Particularly, positions of the sensor accommodating units on the upper garment part and trousers part respectively correspond to locations of muscles of major muscle groups of the user such that the sensors, or electrodes thereof, of the sensor accommodating units are positioned on the corresponding muscle locations of the user when the user wears the personal training device, thereby allowing a posture of the user and a muscle activity of the main muscle groups to be monitored during the user doing an exercise.

In certain embodiments, the upper garment part and trousers part are separate garment articles, are collectively formed as a one-piece garment, or are formed from plural straps. The upper garment part and trousers part are tight-fitting or skin-tight.

In certain embodiments, the upper garment part, trousers part and sensor accommodating units are made of one or more fabrics. A main fabric selected among the one or more fabrics and used for forming the upper garment part and trousers part is tricot knitted. A main fabric selected among the one or more fabrics and used for forming the sensor accommodating units is warp-knitted stretch mesh.

In certain embodiments, the sensors include surface electromyography sensors and inertial measurement unit sensors. The muscles of the major muscle groups include upper trapezius, triceps, erector spinae, biceps femoris, pectoralis major, biceps, rectus abdominis, and rectus femoris.

In certain embodiments, the sensor accommodating units include 14 units for accommodating 16 sensors.

In certain embodiments, the individual sensor accommodating unit is realized as a pocket sewn, snap-attached, or affixed, to the upper garment part or the trousers part.

In certain embodiments, the pocket is a Type-1 pocket for accommodating a single sensor. The Type-1 pocket has a length and a width given by L1=(a+c)×p and W1=(b+2c)×p′ where: L1 is the length of the Type-1 pocket; W1 is the width of the Type-1 pocket; a, b and c are length, width and depth of the accommodated sensor, respectively; p is between 80% and 90% inclusively; and p′ is between 60% and 65% inclusively.

The Type-1 pocket may include an inner layer opening. The inner layer opening is rectangular, oval or square in shape, or conforms to a shape of the accommodated sensor. The inner layer opening has a length and a width given by L3=d+s and W3=e×3 where: L3 is the length of the inner layer opening; W3 is the width of the inner layer opening; d is a sum of lengths of all electrodes in the accommodated sensor; s is between 2 mm and 4 mm inclusively; and e is a common width of the electrodes.

In certain embodiments, the pocket is a Type-2 pocket for accommodating two sensors. The Type-2 pocket has a length and a width given by L2=(a+c)×p and W2=[(2b+2c)+q]×p′ where: L2 is the length of the Type-2 pocket; W2 is the width of the Type-2 pocket; a, b and c are length, width and depth of an individual accommodated sensor, respectively; p is between 80% and 90% inclusively; p′ is between 60% and 65% inclusively; and q is between 5 mm to 10 mm inclusively.

The Type-2 pocket may include an inner layer opening. The inner layer opening is rectangular, oval or square in shape, or conforms to a shape of the individual accommodated sensor. The inner layer opening has a length and a width given by L4=d+s and W4=e×3+f×2 where: L4 is the length of the inner layer opening; W4 is the width of the inner layer opening; d is a sum of lengths of all electrodes in the individual accommodated sensor; s is between 2 mm and 4 mm inclusively; and e is a common width of the electrodes.

In certain embodiments, the personal training device further comprises a wire opening located on an outside part of the pocket and spaced from a sensor insertion opening of the pocket by 1 cm to 2 cm. The wire opening is parallel to a side edge of the pocket or angled to a side of the pocket to facilitate placement of sensor electrodes through the wire opening.

The personal training device based on the AI-assisted wearable sensor developed by the present invention can simultaneously monitor the posture, muscle activity and muscle fatigue in real time during the user doing an exercise. Furthermore, according to the collected data, the personal training device uses an AI algorithm developed by the present invention to evaluate sporting performance, thereby providing real-time feedbacks to improve training efficiency and reduce injury risk.

A second aspect of the present invention is to provide a control device for processing data generated by a personal training device. The control device comprises an input unit, a database, a data preprocessing unit; a training analysis unit and an output unit. The input unit is used for receiving detected-signal data from sensors on the personal training device and inputting a user profile of a user of the personal training device. The database is used for storing the received detected-signal data and the user profile. The data preprocessing unit is used for cleaning and preprocessing the detected-signal data. The training analysis unit is used for executing an artificial intelligence algorithm to perform fatigue analysis on the user's movement and providing training load recommendations based on the user profile and the detected-signal data. An output unit is configured to output an estimated remaining number of repetitions of an exercise to be performed by the user, a training load suggestion given by the training analysis unit, or a number of repetitions of the exercise completed by the user.

In certain embodiments, the control device further comprises a coaching module having a graphical user interface (GUI). The GUI is used for displaying training information and real-time visual feedback of the user's movement, tutorial videos for different types of exercise as stored in the device's exercise library, as well as used for providing audio guidance and feedback on the exercise in real time.

In certain embodiments, the training analysis unit comprises a first deep neural network unit and a second deep neural network unit. The first neural network unit is configured to estimate the user movement according to a current muscle activation signal. The second deep neural network unit is configured to suggest an optimal training load to the user according to the current muscle activation signal and the estimated remaining number of repetitions.

In certain embodiments, the control device further comprises a posture detection model unit and a machine learning posture classifier. The posture detection model unit is configured to receive a signal of the user's human body detected from a camera input unit, and to generate a human body marker based on the detected signal of the user's human body. The machine learning gesture classifier is configured to calculate a vector representing the user's ongoing exercise program and a motion state from the human body marker generated by the posture detection model unit, whereby the control device is realized as an artificial intelligence fitness training system.

In certain embodiments, the data preprocessing unit includes a signal preprocessing unit for performing one or more of the following preprocessing functions on the detected-signal data: bandpass filtering; highpass filtering; lowpass filtering; root mean square calculation; moving average calculation; mean absolute value calculation; median frequency calculation; data segmentation; data normalization; and data anomaly identification.

A third aspect of the present invention is to provide a personal training system.

The personal training system comprises any of the embodiments of the personal training device and any of the embodiments of the control device.

In certain embodiments, the personal training system further comprises a machine learning server, an application server and a database server. The machine learning server is used for executing artificial intelligence algorithms for machine learning. The application server is used for storing one or more application programs of the control device, allowing the user to download the one or more application programs. The database server is used for storing collected data, providing data to the artificial intelligence algorithm and supporting data analysis.

Other aspects of the present disclosure are disclosed as illustrated by the embodiments hereinafter.

### BRIEF DESCRIPTION OF THE DRAWINGS

FIG. 1A shows a schematic view of a front side of a personal training device in accordance with a first embodiment of the present invention, where the personal training device is of men's style.

FIG. 1B shows a schematic view of a back side of the personal training device of FIG. 1A.

FIG. 1C shows a schematic view of a front side of a personal training device in accordance with a second embodiment of the present invention, where the personal training device is of women's style.

FIG. 1D shows a schematic view of a back side of the personal training device of FIG. 1C.

FIG. 2A shows a front view of the personal training device designed for men.

FIG. 2B shows a back-side view of the personal training device of FIG. 2A.

FIG. 3A shows a front view of the personal training device designed for women.

FIG. 3B shows a back-side view of the personal training device of FIG. 3A.

FIG. 4A depicts a top view of a Type-1 pocket for insertion of a single sensor in the personal training device.

FIG. 4B depicts a side view of the Type-1 pocket of FIG. 4A.

FIG. 5 shows a Type-2 pocket for insertion of two sensors in the personal training device.

FIG. 6 shows a Type-3 pocket that can be used for the pectoralis major sensor in the personal training device.

FIG. 7A depicts a dimension of the Type-1 pocket of the personal training device.

FIG. 7B depicts a dimension of the Type-2 pocket of the personal training device.

FIG. 7C depicts a schematic diagram of sensors and their dimensions in the personal training device.

FIG. 8A shows a rectangular opening dimension for the Type11 pocket in the personal training device.

FIG. 8B shows a rectangular opening dimension for the Type-2 pocket in the personal training device.

FIG. 8C shows a schematic diagram of sensors and their dimensions in the personal training device.

FIG. 9A depicts four pairs of muscles on the front side of the human body, where the four muscle pairs are monitored by wearable sensors in the personal training device.

FIG. 9B depicts four pairs of muscles on the back side of the human body, where the four muscle pairs are monitored by wearable sensors in the personal training device.

FIG. 10A illustrates positions of the sensors installed on the front side of the human body, the sensors being of the personal training device, the positions being optimized after multiple rounds of wear testing.

FIG. 10B illustrates positions of the sensors installed on the back side of the human body, the sensors being of the personal training device, the positions being optimized after multiple rounds of wear testing.

FIG. 11A shows a schematic block diagram of a control device in the personal training system.

FIG. 11B shows a schematic block diagram of one embodiment of the first deep neural network unit used in the control device of FIG. 11A.

FIG. 12A illustrates an exercise included in a resistance training program suitable for the personal training device, where the exercise is Step Up for 3 minutes.

FIG. 12B illustrates an exercise included in the resistance training program suitable for the personal training device, where the exercise is Chest Press for 10 times×3 sets.

FIG. 12C illustrates an exercise included in the resistance training program suitable for the personal training device, where the exercise is Two arm rolls for 10 times×3 sets.

FIG. 12D illustrates an exercise included in the resistance training program suitable for the personal training device, where the exercise is Bent-over Biceps curl for 10 reps×3 sets.

FIG. 12E illustrates an exercise included in the resistance training program suitable for the personal training device, where the exercise is Lying triceps extension for 10 reps×3 sets.

FIG. 12F illustrates an exercise included in the resistance training program suitable for the personal training device, where the exercise is Sit-up for 10 times×3 sets.

FIG. 12G illustrates an exercise included in the resistance training program suitable for the personal training device, where the exercise is Russian Twist for 10 times×3 sets.

FIG. 12H illustrates an exercise included in the resistance training program suitable for the personal training device, where the exercise is Split Squat for 10 times×3 sets×2 legs.

FIG. 12I illustrates an exercise included in the resistance training program suitable for the personal training device, where the exercise is Plank for 30 seconds×2 times.

FIG. 13 shows a schematic diagram of the personal training system.

FIG. 14A shows a schematic diagram of a GUI of a coaching module in the personal training system.

FIG. 14B is a schematic diagram of a user wearing the personal training device to do exercise and a corresponding diagram of sensors on the user in the GUI of FIG. 14A.

FIG. 15A depicts a first smart garment in the prior art.

FIG. 15B depicts a second smart garment in the prior art.

FIG. 15C depicts a third smart garment in the prior art.

Skilled artisans will appreciate that elements in the figures are illustrated for simplicity and clarity and have not necessarily been depicted to scale.

### DETAILED DESCRIPTION

As used herein, “upper garment part” means a clothing item, or a portion thereof, that is intended to be worn on a torso of a person. Examples of an upper garment part include a shirt, a sweater, a coat and a jacket, as well as an upper portion of a one-piece clinging garment that covers the person from his/her shoulder to ankles.

As used herein, “trousers part” means a clothing item, or a portion thereof, that is shaped like a pair of trousers and is intended to be worn below a waist of a person for covering at least a hip part of the person. Examples of a trousers part include a pair of trousers and a pair of shorts, as well as a lower portion of a one-piece clinging garment that covers the person from his/her shoulder to ankles.

Disclosed herein are a personal training device, a control device for processing data generated by the personal training device, and an AI-assisted personal training system that includes the personal training device and the control device.

The personal training device comprises an upper garment part, a trousers part, and plural sensor accommodating units. Both of the upper garment part and trousers part are used for wearing by a user. The sensor accommodating units are distributed on the upper garment part and trousers part. Plural sensors are installed in the sensor accommodating units such that an individual sensor accommodating unit is equipped with one or more of the sensors. Positions of the sensor accommodating units on the upper garment part and trousers part respectively correspond to locations of muscles of major muscle groups of the user such that the sensors, or electrodes thereof, of the sensor accommodating units are positioned on the corresponding muscle locations of the user when the user wears the personal training device. Thereby, it allows a posture of the user and a muscle activity of the main muscle groups to be monitored during the user doing an exercise.

The control device comprises an input unit, a database, a data preprocessing unit, a training analysis unit, and an output unit. The input unit is used for receiving detected-signal data from sensors on the personal training device and inputting a user profile of a user of the personal training device. The database is used for storing the received detected-signal data and the user profile. The data preprocessing unit is used for cleaning and preprocessing the detected-signal data. The training analysis unit is used for executing an artificial intelligence algorithm to perform fatigue analysis on the user's movement and providing training load recommendations based on the user profile and the detected-signal data. The output unit is configured to output an estimated remaining number of repetitions of an exercise to be performed by the user, a training load suggestion given by the training analysis unit, or a number of repetitions of the exercise completed by the user.

Specific embodiments of the present invention will be described in further detail below with reference to the accompanying drawings.

The AI-assisted personal training system of the present invention may include the wearable personal training device, the control device, and a server.

The wearable personal training device may have the shape of a garment to facilitate donning and doffing by a user. For example, the wearable personal training device includes a top (or T-shirt) and shorts; alternatively, a plurality of straps, such as waist belts, shoulder straps, girdle straps, leggings, shin guards, etc., which can be interconnected for ease of use. The wearable personal training device includes a plurality of sensors, and the plurality of sensors can be respectively mounted on the wearable personal training device through, for example, pockets or Velcro, so that after the user wears the wearable personal training device, the, the plurality of sensors or their electrodes are respectively located at positions corresponding to different muscle parts of the user.

The control device may be implemented as a stand-alone hardware device, or as application software to be installed in a mobile phone or other portable communication device. Alternatively, the control device may be a computer program product installed in a computer with a Windows or iOS operating system. The control device may be wired to the sensor via a wire, or wirelessly connected to the sensor via WiFi or Bluetooth, or the like. When the user performs exercise or exercises after putting on the wearable personal training device, the control device can simultaneously monitor the user's posture and muscle activity during the exercise through the plurality of sensors. Preferably, the monitoring may be performed in real time. The AI algorithm module built into the control device evaluates the user's training performance based on the sensor data, and can provide real-time feedback and suggestions to the user through a GUI and/or audio feedback.

The servers may include machine learning servers, application servers, and database servers. The Inventors have developed a centralized database with multiple uses that can: store collected data; provide data to AI algorithms; and support data analysis. All data collected, used and processed are stored in a database. The data can include, for example, user credentials, training records, sEMG signal readings, and some personalization parameters optimized by AI algorithms.

The individual components of the AI-assisted personal training system of the present invention will be described in detail below with reference to the accompanying drawings.

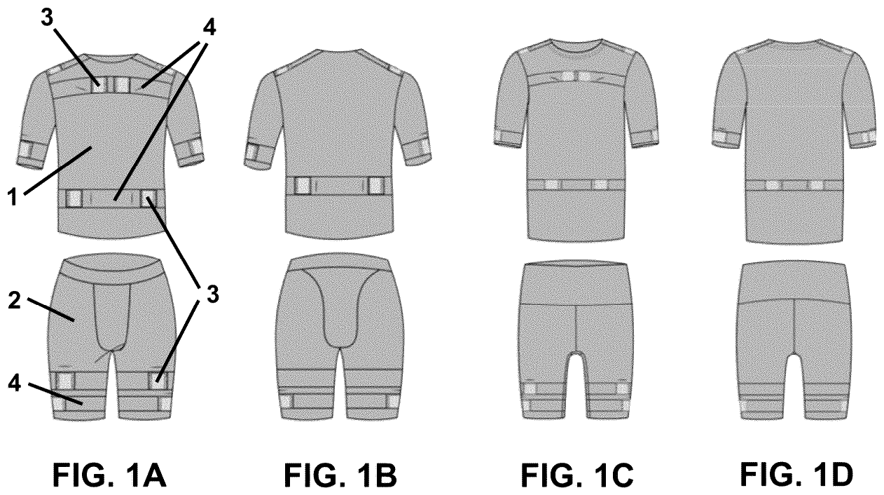

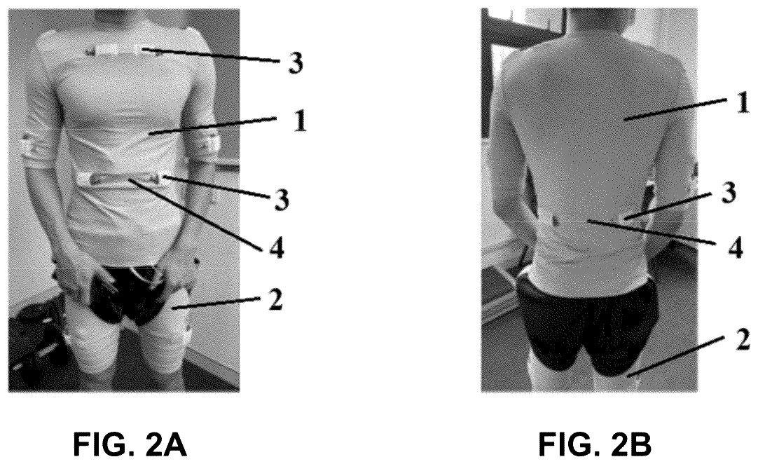

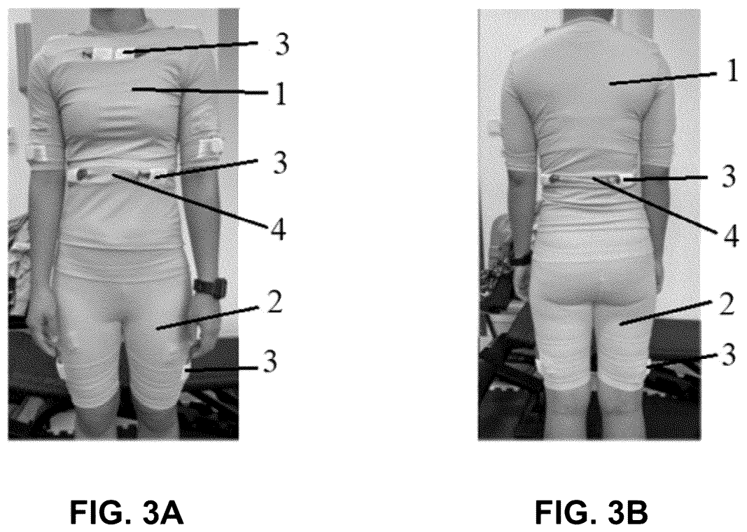

The wearable personal training device of the present invention is based on AI-assisted sensors. One embodiment of the wearable personal training device may include a top 1 and a pair of shorts 2 (or trousers), as shown in FIGS. 1A-3B. FIGS. 1A-1D show schematic diagrams of the front and back of the personal training device of the present invention, wherein FIGS. 1A and 1B represent men's styles, and FIGS. 1C and 1D represent women's styles; FIGS. 2A and 2B respectively show the user's front and back views of donning the men's personal training device of the present invention. FIGS. 3A and 3B show front and back views, respectively, of a user donning the women's personal training device of the present invention.

The top 1 and the shorts 2 each have a plurality of sensor accommodating units, such as pockets 3, to accommodate up to 16 sensors, and the installation positions of these sensors can respectively correspond to the skin of up to 16 different body parts. The tops and shorts may also be constructed as a one-piece garment comprising an upper garment part and a trousers part, or in other styles, such as multiple straps 4 for easy attachment and donning, with pockets, locks or Velcro Removably mounts the sensor and is easy to wear on the user. Alternatively, the sensor may be fixed to the personal training device rather than being detachable.

According to the invention, the personal training device is designed for the combination of sensors 5 to detect the muscle activity and body movement of the user by means of said sensors. The sensor may also be called an external sensing device, and is used to send or transmit the detected signal to an external device such as a control device. The personal training device can be designed to include men's and women's styles. See FIGS. 2A-2B and FIGS. 3A-3B, respectively. Each style can be divided into three sizes, namely, small (S), medium (M) and large (L), or may have more sizes for users of different heights.

Preferably, the personal training device is designed to be tight-fitting or skin-tight to ensure that the sensor mounted in the sensor accommodating unit 3 can be in close contact with the user's skin and that the sensor is on the skin when the user is exercising has the smallest displacement. Preferably, the personal training device can be designed as a short-sleeved t-shirt and shorts to provide maximum flexibility for the user to exercise and easily insert the sensor. There may be as many as 16 or more pockets on the personal training device, each for insertion of sensors.

The top 1 and shorts 2 of the personal training device may be made of, for example, nylon spandex fabric. For example, warp-knitted tricot fabrics have excellent stretch, providing a good fit and free body movement for fitness training, ensuring accurate and stable muscle activity and body movement detection. The fabric typically has good breathability and moisture absorption to maintain good thermophysiological and ergonomic wearing comfort when the sensor is mounted on the personal training device and the user is training.

The pockets used to accommodate the sensors are usually made of fabric that is elastic for easy insertion of the sensor and has see-through properties for monitoring the condition of the sensor. Warp-knitted powernet fabrics are an example of this requirement. Table 2 lists an example of fabric properties for suggested fabrics.

TABLE 2
Examples of fabric composition specifications for
personal training devices of the present invention.

| Fabric | | | Weight |
|---|---|---|---|
| Type | Construction | Composition | (g/m) |
| Main fabric | Warp knitted tricot | 79% nylon, | 180 |
| | | 21% Spandex | |
| Pocket | Warp knitted powernet | 74% nylon, | 160 |
| fabric | (stretch mesh) | 26% Spandex | |

The sEMG and inertial measurement unit (IMU) sensors on the personal training device of the present invention can be any sEMG sensor that meets the following specification requirements: (1) has a communication interface available; (2) is capable of acquiring sEMG signals at a sampling rate of >1000 Hz without notch filtering (3) capable of collecting 9-axis IMU data at a sampling rate of >200 Hz, including data from 3 accelerometers, 3 gyroscopes, and 3 magnetometers; (4) all sensor data are time-synchronized; (5)) The sensor is connected to a pair of disposable electrodes by wires, which can be between 3 and 5 inches in length. The personal training device of the present invention can use state of the art sEMG sensors, such as the Noraxon Ultium EMG sensor system, which includes a receiver and 16 wireless surface EMG sensors with an internal IMU.

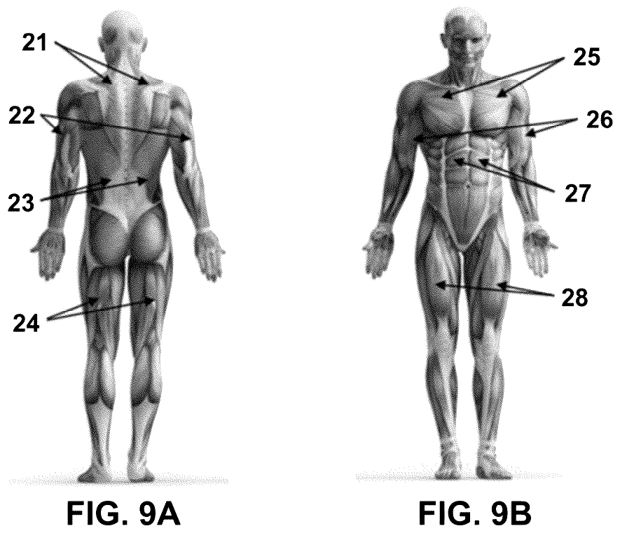

The personal training device shown in FIGS. 1A-3B has 14 pockets that can accommodate 16 sensors. According to certain embodiments of the present invention, these sensors are installed at positions corresponding to 8 pairs of muscles of the human body, respectively, to collect sEMG and IMU signals of these muscles when the user exercises. For example, FIGS. 9A and 9B show eight pairs of muscles monitored by sensors in accordance with the present invention: upper trapezius, triceps, erector spinae, biceps femoris, pectoralis major, biceps, rectus abdominis, rectus femoris. The pockets are designed to hold and stabilize the sensor during movement. They can be sewn horizontally to the fabric surface to minimize the number of fabric surfaces and seams. The insertion opening of the pocket may be open or bound, or may have buttons or Velcro or the like to close the insertion opening after insertion of the sensor.

Regarding the position of the sensor accommodating unit relative to the body of the user who wears the personal training device, that is, the position of the above-mentioned sensor installed on the user's body, the Inventors have conducted long-term in-depth research and a large number of experiments, Finally, the sensor accommodating unit (that is, the sensor) is selected to be placed at the position corresponding to the above-mentioned eight pairs of muscles. The reasons mainly include: (1) The eight pairs of muscles are superficial layers that can be detected by sEMG; muscles, and (2) the eight pairs of muscles are the main muscle groups that are often trained in resistance training. Of the eight pairs of muscles, the biceps and triceps are responsible for flexion and extension of the elbow. The rectus femoris and biceps femoris are responsible for flexion and extension of the knee. The pectoralis major is responsible for flexion, adduction, and internal rotation of the arm. The rectus abdominis and erector spinae are very important postural muscles. The rectus abdominis controls trunk flexion, and the erector spinae extends and rotates the spine. The upper traps support the weight of the arms and stretch the neck. Another reason to choose a position that corresponds to the upper trapezius is that most people use it unintentionally when it should be relaxed. Therefore, according to the present invention, by placing the sensors at positions corresponding to these muscles, it is possible to more accurately detect the motion of the user and provide feedback to the user.

In contrast, the Hexoskin and Nadi X yoga pants listed in Table 1 do not measure muscle activity at all. Although the Athos training system can measure more muscles than the present invention, its performance analysis is completely different from that of the present invention. The Athos training system compares measured sEMG data, such as muscle activation levels, to reference values based on the target muscle being trained. For example, for a squat, the rectus femoris should reach 40% activation. The present invention further considers muscle fatigue by using variables in the frequency domain on top of the muscle activation level determined from the magnitude of the sEMG signal. Therefore, the detection results of the present invention are more reliable because the magnitude of the sEMG signal can be severely affected by the skin condition, especially in the presence of sweat, which is often the case when a user is exercising. Furthermore, the present invention uses a camera or camera to monitor the user's gestures and movements, improving the accuracy of motion detection compared to using wearable sensors alone.

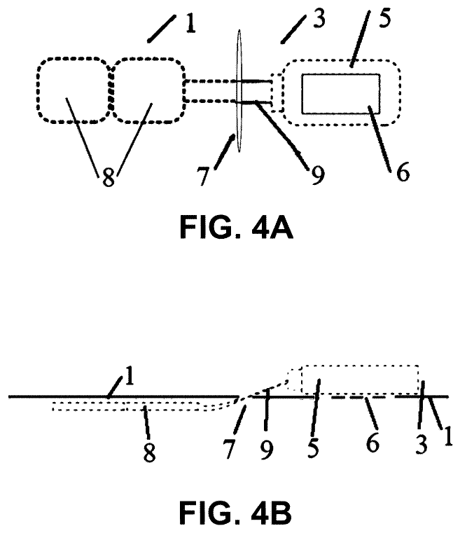

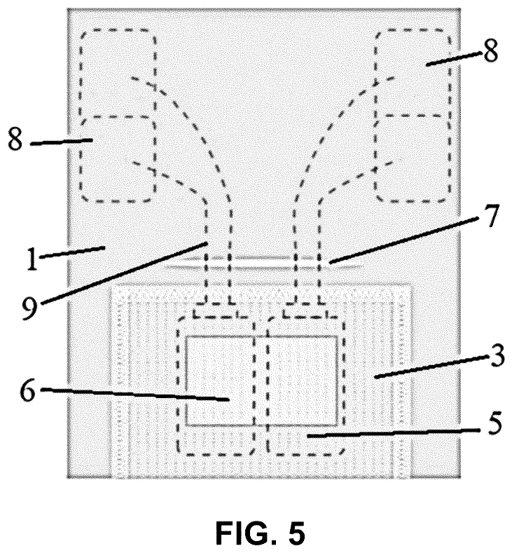

FIGS. 4A and 4B illustrate top and side views, respectively, of a Type-1 pocket design for insertion of a single sensor in the personal training device of the present invention. FIG. 5 shows a Type-2 pocket design for insertion of 2 sensors in the personal training device of the present invention. FIG. 6 shows a Type-3 pocket design that can be used for the pectoralis major sensor in the personal training device of the present invention. FIGS. 4A, 5 and 6 respectively show a front view of the design of the pocket 3, while FIG. 4B is a cross-sectional or side view of the pocket 3 of the Type-1 design, and a cross-sectional view of the pocket design in FIGS. 5 and 6 similar to FIG. 4B. For example, the sensor 5 may be a surface electromyography sensor, and the electrode 8 may be a surface electromyography electrode.

According to the present invention, the personal training device may comprise two types of pockets.

Type-1 design—for single sensor insertion. FIGS. 4A and 4B show a Type-1 pocket that facilitates the insertion of a single sensor 5. There may be, for example, 12 such pockets 3 on the training device. They are stitched horizontally or vertically into positions corresponding to the designated body parts, respectively. Pockets at locations corresponding to the torso of the body are sewn horizontally; while pockets at designated locations corresponding to upper and lower extremities may be sewn vertically along the sleeves and pants.

Type-2 design—for dual sensor insertion. FIG. 5 shows an example of a Type-2 pocket 3 into which sensors 5 for measuring biceps and triceps can be inserted. This type of pocket can be sewn on the sleeve to hold the two sensors 5 (for measuring the biceps and triceps respectively) together. A Type-2 pocket may be a single pocket into which two sensors may be inserted; alternatively, a Type-2 pocket may be divided into two pockets that are placed side by side, each pocket may accommodate a sensor and may share a single insertion opening, or each one has an insertion port.

Type-3 design—for single sensor insertion. It is actually a variant of the Type-1 design, which differs from the Type-1 design in that the mounting position and/or orientation of the electrodes 8 and the orientation of the linear openings 7 have been modified to suit different bodies or muscle areas. An example of a pocket design for a sensor for the pectoralis major is shown in FIG. 6.

As shown in FIGS. 4A to 6, the inner layer of each pocket 3 may have an inner layer opening 6, which may be, for example, rectangular, square, circular or oval. There may also be wire openings 7 beside or inside the pocket 3 to allow wires 9 to pass through the wire openings 7 to connect the sensor 5 to its electrodes 8, which wire openings 7 may be linear, such as slit. The wire opening 7 may be spaced about 1-2 cm from the side of the pocket 3 or the sensor insertion opening, such as 1.5 cm, and the length of the wire opening 7 may be slightly larger than the size of the electrode 8 of the sensor 5 (e.g., the length of the side). or diameter) to facilitate placement of the electrodes 8 and wires 9 of the sensor 5 through the wire openings 7. In FIGS. 4A-6, the pockets may be of a wrap design, so that the wires 9 in these figures may be arranged within the wrap. The electrodes 8 may include positive and negative electrodes, which may be respectively attached to the user's skin corresponding to the position of the muscle to be detected. For example, referring to FIG. 6, the wire opening 7 of the pectoralis major sensor 5 is cut obliquely with respect to the pocket 3 so that the electrodes 8 can be placed at an angle relative to the sensor 5, e.g., arranged perpendicular to the sensor 5.

FIG. 4B is a cross-sectional view of the pocket 3 of the Type-1 design, wherein the electrodes 8 of the sensor 5 are placed on the inside of the clothing material of the top 1 (or shorts 2), against the skin of the user, and the wires 9 pass through the wire openings 7 to connect the electrodes 8 to the user's skin. The sensor 5 is connected; the sensor 5 is installed in the pocket 3, and the bottom surface of the sensor 5 is in contact with the user's skin through the inner layer opening 6 of the pocket 3.

It is understood that the inner layer opening 6 and wire opening 7 are optional; different sensors 5 may require inner layer opening 6 and wire opening 7 of different shapes or sizes, or may not require inner layer opening 6 and wire opening 7. In addition, both the inner layer opening 6 and the wire opening 7 may be located in the inner layer of the pocket 3; alternatively, the inner layer opening 6 may be used as a wire opening, so that a separate wire opening 7 may not be required.

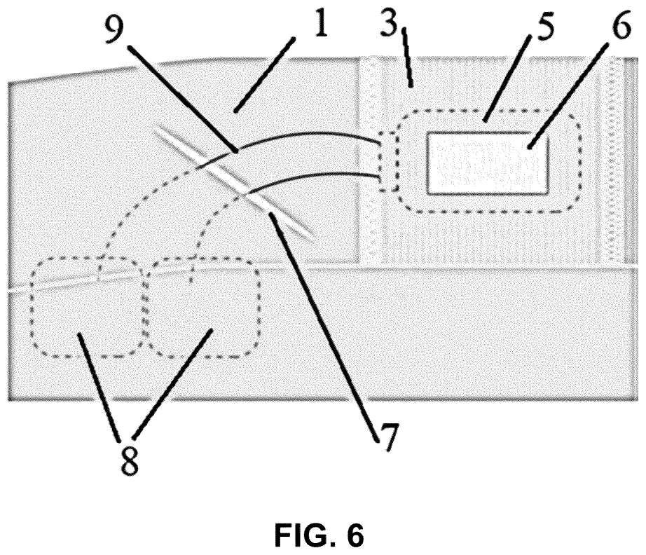

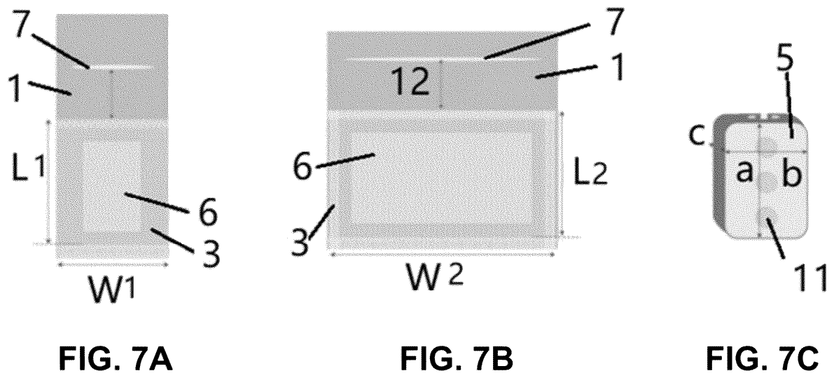

FIGS. 7A and 7B show the dimensions of the Type-1 pocket design and the Type-2 pocket design in the personal training device of the present invention, respectively, and FIG. 7C shows a schematic diagram of the sensor 5 and its dimensions in the personal training device of the present invention.

In order to fix the position of the sensor during the movement of the user, the pocket 3 can generate sufficient tension or restraint force on the sensor 3 after the sensor 5 is inserted into the pocket 3. Thus, the size of the pocket may depend on the size of the sensor used. FIGS. 7A and 7B show schematic diagrams of the dimensions of Type-1 pockets and Type-2 pockets, respectively, and FIG. 7C is a schematic diagram of sensors and their dimensions. It is denoted that the length, width and depth of the sensor 5 are a, b, and c, respectively, and the length and width of the pocket 3 are L and W, respectively. Generally, it can be considered that the pocket 3 is substantially planar, and its thickness can be ignored. For a three-dimensionally designed pocket, the length and width after flattening can correspond to the length and width in the following formulas, respectively. For a Type-1 pocket, its length L1 may typically be about 80-90% of the sum of the sensor length and depth, i.e. L1=(a+c)×p, where p is about 80-90%; the width of the pocket W1 can be about 60%-65% of the sum of the sensor width and twice the depth, i.e. W1=(b+2c)×p, where p is about 60%-65%. For a Type-2 pocket, pocket length L2 is calculated in the same way as for the Type-1 pocket, while pocket width W2 is 60%-65% times the sum of twice the sensor width, twice the sensor depth, plus a margin of approximately 5-10 mm (millimeters), or W2=[(2b+2c)+q]×p, where p is about 60%-65%, and q is about 5-10 mm (millimeters).

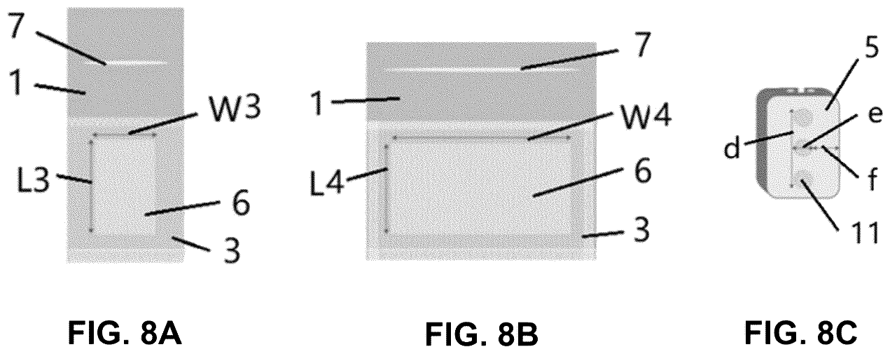

FIGS. 8A and 8B show the dimensions of the openings in the pockets for Type-1 and Type-2 pocket designs, respectively, in the personal training device of the present invention, and FIG. 8C shows a schematic diagram of the sensors and their dimensions in the personal training device of the present invention.

The opening 6 in the pocket 3 can be in common shape such as rectangle, circle, ellipse, square, etc., or the same shape as the sensor 5, so as to facilitate the manufacture and installation of the sensor 5. As shown in FIGS. 8A-8B, the length L3 of the rectangular opening of the Type-1 pocket can be the length (d) of all electrodes in the sensor plus a margin of 2-4 mm, i.e. L3=d+s, s being about 2-4 mm; the width W3 of the rectangular opening of the Type-1 pocket can be three times the electrode width (e), i.e. W3=e×3. For the Type-2 pocket, the length L4 of the rectangular opening is calculated to be the same as for the Type-1 pocket, while the width W4 of the rectangular opening can be four times of the electrode diameter (e) plus twice the sensor base width (f) from electrode to side. It follows that W4=e×4+f×2.

One aspect of the important contribution of the personal training device of the present invention is the optimization of the positions of the various sensors on the personal training device, which are particularly advantageous for detection of specific movements or forms of exercise performed by the user.

For example, in order to detect the EMG signal of the target muscle, a pair of EMG electrodes must be mounted to the muscle belly position of the target muscle, and then connected to the EMG sensor through a pair of short wires. In order to collect more accurate and stable body part IMU data, the EMG sensor (where the IMU is located) is preferably placed parallel and/or perpendicular to the body part.

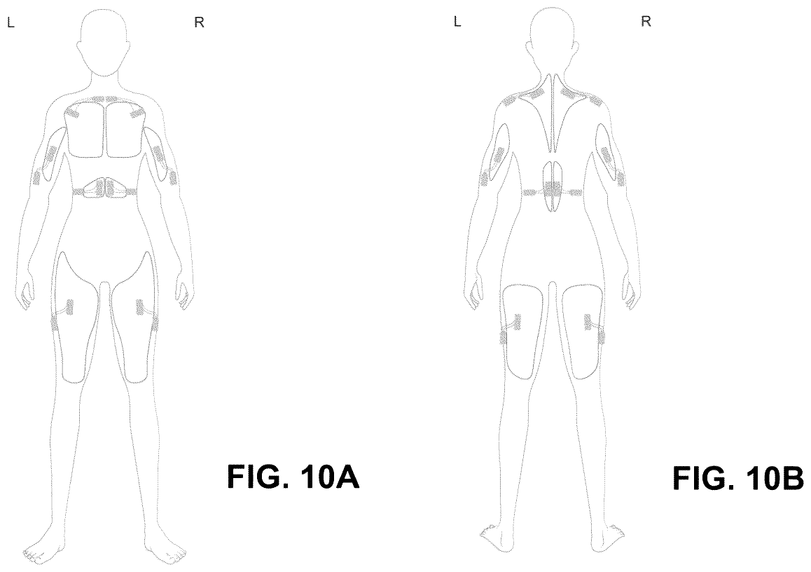

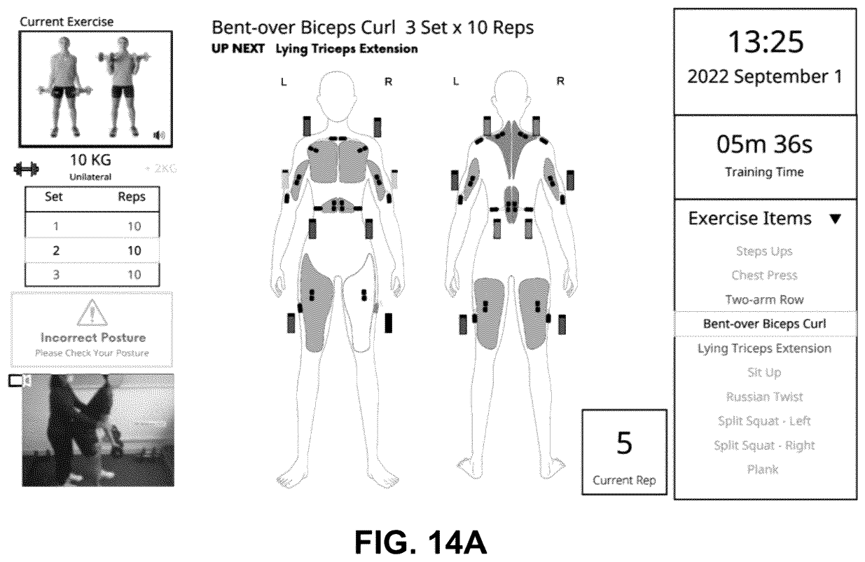

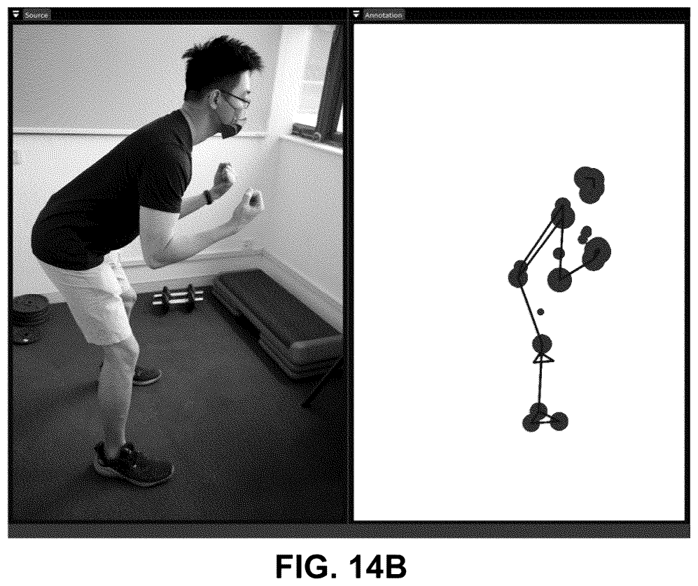

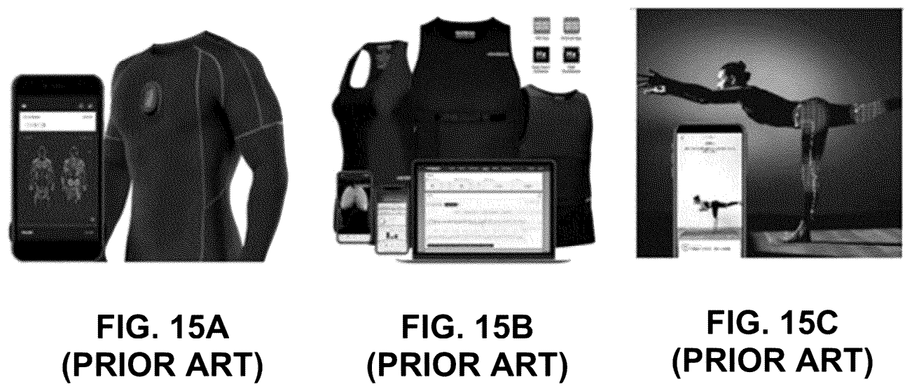

Preferably, the personal training device of the present invention allows for the simultaneous monitoring of 8 pairs of muscles in the user's body, which are the major muscle groups of the user's body. FIGS. 9A and 9B show, respectively, eight pairs of muscles in the front and back of the human body, including: upper trapezius, triceps, erector spinae, femoris, monitored by wearable sensors in the personal training device of the present invention. Biceps, pectoralis major, biceps, rectus abdominis, rectus femoris. FIGS. 10A and 10B respectively show schematic diagrams of the positions of the sensors in the front and back of the human body in the personal training device of the present invention optimized after multiple rounds of wearing tests, respectively, these positions correspond to the body positions of the above-mentioned 8 pairs of muscles, respectively. Eight sensors are shown in FIGS. 10A and 10B each, and each sensor includes two electrodes connected by wires, where the sensors are represented by rectangular white blocks, the electrodes are represented by square white blocks, and the contours of the human body and individual muscles are indicated by white lines. Each sensor is located near the corresponding muscle, and the electrode of the sensor is located at the position of the muscle belly in the contour of the corresponding muscle, so as to monitor the electromyogram of the corresponding muscle when the user is exercising. FIGS. 10A and 10B show the outline of the human body with a white background. Alternatively, the background may be set to black or other colors, or individual muscles and individual sensors are shown in color for user convenience. Similarly, FIGS. 14A and 14B below can also be provided as schematic diagrams in various colors or colors.

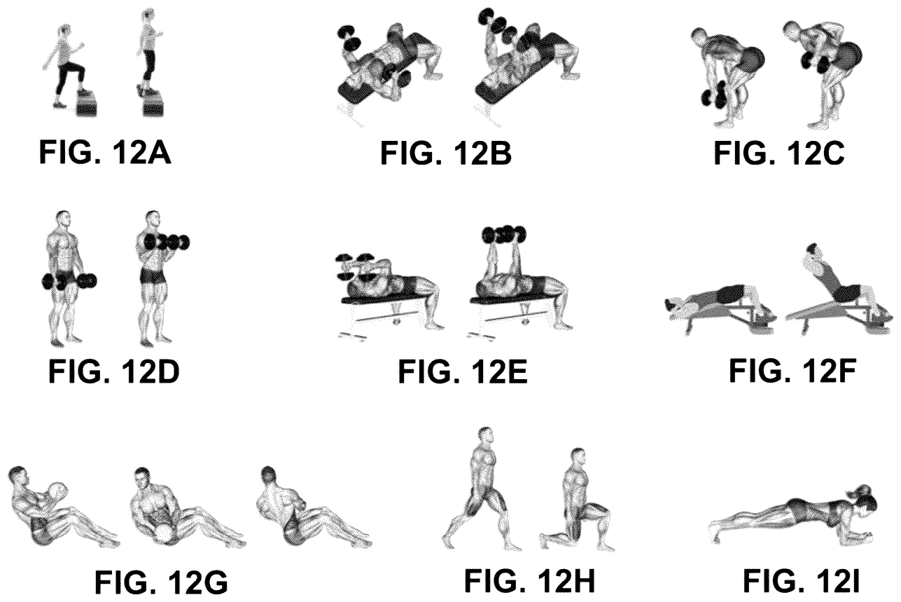

The personal training device of the present invention can perform motion recognition and abnormality detection for the user when the user is exercising after wearing. In order to train major muscle groups, the present invention designs a whole-body resistance training program including 10 exercises. FIGS. 12A to 12I respectively show schematic diagrams of the nine exercises included in the resistance training program suitable for the personal training device of the present invention, including: FIG. 12A: Step Up for 3 minutes; FIG. 12B: Chest Press (Chest Press) 10 times×3 sets; FIG. 12C: Two arm rolls (Two arm roll) 10 times×3 sets; FIG. 12D: Bent-over Biceps curl 10 reps×3 sets; FIG. 12E: Lying triceps extension 10 reps×3 sets; FIG. 12F: Sit-up) 10 times×3 sets; FIG. 12G: Russian Twist 10 times×3 sets; FIG. 12H: Split Squat 10 times×3 sets×2 legs; FIG. 12I: Plank 30 seconds×2 times. These exercise items and the number of sets and/or times are only examples, and the user may also appropriately perform other exercise items and/or manners, or exercise with different sets and/or times.

Different exercise modes generate different muscle activation patterns for the user, and these exercise modes can be distinguished in the AI algorithm of the control device of the personal training system of the present invention, i.e, motion recognition. One of the goals of the AI algorithm is to identify the type of movement in real time. The AI algorithm can receive as input readings from 16 sEMG sensors, with or without camera feeds, and then use K-means (k-means) clustering to identify exercise patterns or exercise types. In addition to motion recognition, the algorithm is also able to deduce the current position of the user performing the motion and anomalies, such as incorrect or overuse of certain muscle groups and underuse of stabilizers (if any). A real-time warning is issued to the user if the abnormal situation may increase the risk of injury to the user. For exercises other than those shown in FIGS. 12A-12I, the present invention may be extended to provide different solutions for sensors and their locations, which also remain within the scope of the present invention.

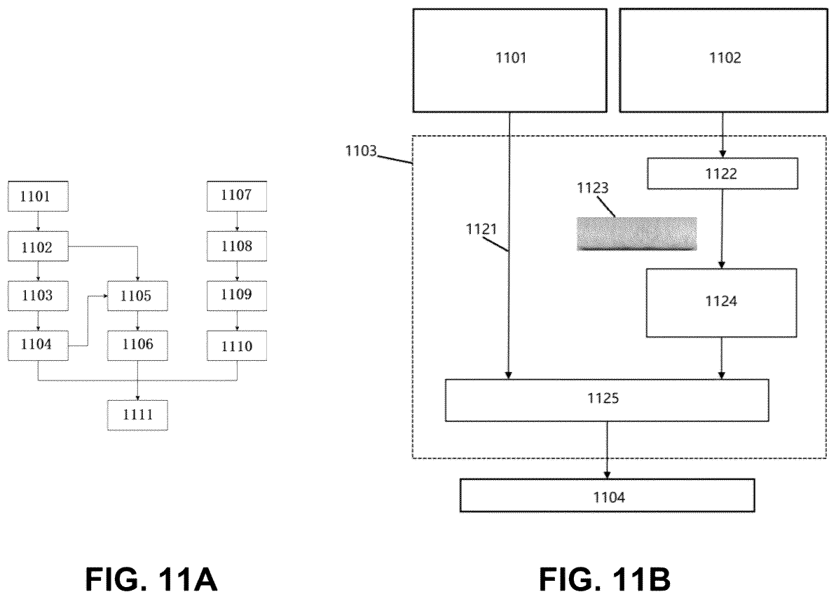

FIG. 11A shows a schematic block diagram of the control device in the personal training system of the present invention. The control device is configured to: (1) receive information characterizing a user profile of the user, (2) receive data from an external sensor system, (3) calibrate the sensor data according to the user profile, (4) preprocess sensor data, (5) identify current exercise progress from sensor data (using the machine learning method of the present invention), (6) detect abnormalities in muscle utilization (using the machine learning method of the present invention), (7) detect muscle fatigue (adopting the machine learning method of the present invention), (8) update the database server, (9) recommend the training load for the next training session, and (10) provide a coaching module for providing visual and audio feedback to the user.

The control device of FIG. 11A is an AI fitness training system 1111, which adopts a method based on machine learning for data collection and preprocessing. As shown in FIG. 11A, the AI fitness training system 1111 of the present invention may include a signal input unit 1101, a signal preprocessing unit 1102, a first deep neural network unit 1103, a first output unit 1104, a second deep neural network unit 1105, a second output unit 1106, a camera input unit 1107, a posture detection model unit 1108, a machine learning pose classifier 1109, and a third output unit 1110.

(1) Signal Input Unit 1101

The signal input unit 1101 may receive signals input from, for example, 16 sEMG sensors, and compose time-series data from the signals reported by the sEMG. The input signals may be, for example, in the order of microvolts (μV). The sEMG sensor may be the aforementioned sensor 5 located on the personal training device worn by the user. The signal input unit 1101 may be coupled to the sEMG sensor wirelessly or wired. For example, the signal input unit 1101 may receive sEMG and IMU data collected by each sensor (e.g., the sensor 5 as described above) as shown in FIGS. 10A and 10B. The sensors may be wireless sensors, for example, with up to 16 sensors organized into 10 channels.

In addition, the signal input unit 1101 may also receive information about the user or user data, such as the user's gender, weight, height, BMI (Body Mass Index), body fat ratio, muscle mass, and exercise selected by the user to be performed, exercise type, etc. Such information or data can be inputted in a prescribed or predetermined format, and can even be directly inputted into the first deep neural network unit 1103 for further processing.

(2) Signal Preprocessing Unit 1102

The signal input unit 1101 transmits the input data to the signal preprocessing unit 1102 for preprocessing to form data in a predetermined format, which is then supplied to other units (for example, the first deep neural network unit 1103 or the second deep neural network unit 1105) for further use. For example, the raw sEMG and IMU data imported from the signal input unit 1101 are cleaned and preprocessed by the signal preprocessing unit 1102. The preprocessing may include (but is not limited to): (1) bandpass, highpass and/or lowpass filtering; (2) rms/moving average/mean absolute/median frequency calculations; (3) data segmentation—which can be done by repetition; (4) data normalization; and (5) data anomaly identification.

(3) The First Deep Neural Network Unit 1103

The signal preprocessing unit 1102 first transmits the preprocessed data to the first deep neural network unit 1103. The first deep neural network unit 1103 is used for executing the AI algorithm of the present invention to perform fatigue analysis on the user's movement, and to estimate the remaining number of repetitions that the user can do according to the current muscle activation signal.

One of the goals of the AI algorithm of the control device is to identify the type of movement in real time. The AI algorithm can receive as input the readings of the 16 sEMG sensors from the signal input unit 1101 (which may or may not be fed with the camera input unit 110), and then use K-means (k-means) clustering to identify the exercise style or type of exercise. In addition to the recognition of the type of exercise, the AI algorithm can also use the first deep neural network unit 1103 to deduce the current position and abnormal situation of the user's exercise, such as the incorrect or overuse of certain muscle groups and the stability of the Inadequate muscle use (if any). A real-time warning is issued to the user if the abnormal situation may increase the risk of injury to the user.

(4) The First Output Unit 1104

The first output unit 1104 represents the estimated number of repetitions remaining, which is an estimate of the number of repetitions the user can complete after the current repetition. It is a positive integer, such as 6, which means the user can perform 6 more repetitions.

(5) The Second Deep Neural Network Unit 1105

The second deep neural network unit 1105 obtains the processed signal data from “signal preprocessing”, and obtains the output from the first output unit 1104. The second deep neural network unit is responsible for executing the AI algorithm of the present invention, suggesting to the user the optimal training load (e.g., in kilograms) based on the current muscle activation signal and the estimated number of remaining repetitions. For example, regardless of the weight of the current training load, its output could be, for example, “20 kg”.

The suggested training load should ideally allow the user to complete the training set with the number of repetitions defined by the training plan of the present invention, and not perform fewer or more repetitions. The recommendations are updated after each training set, so the user is expected to change the training load between training sets if the optimal load is different.

(6) Second Output Unit 1106

The second output unit 1106 represents the user's optimal training load. The recommended training load is expressed in kilograms (kg), and the output for the example is 20 kg. The second output unit 1106 is only updated between training sets.

(7) Camera Input Unit 1107

The camera input unit 1107 represents a video input from the camera. The camera is used to capture the actions of the user, and the user should keep his body in the picture of the camera when performing training.

(8) Posture Detection Model Unit 1108

The output of the camera input unit 1107 is passed to the gesture detection model unit 1108, which receives the user body signal detected from the camera input unit 1107 and generates a body marker based on the user body signal. The gesture detection model unit 1108 may, for example, take a 3-dimensional vector (x, y, z) to output a total of 33 body markers.

(9) Machine Learning Pose Classifier 1109

The machine learning gesture classifier 1109 calculates an 18-dimensional vector (feature vector) from the human body markers output by the gesture detection model unit 1108 to represent the exercise item and the movement state that the user is performing. The vectors can be respectively shown in Table 3 below.

TABLE 3
Vectors representing pose classification.

| No. | Name | Description |
|---|---|---|
| 0 | Left Arm Curl | The distance between the left wrist |
| | | and the left shoulder. |
| 1 | Right arm curl | between the right wrist and the |
| | | right shoulder. |
| 2 | Left Elbow Lateral | The distance between the left elbow |
| | Distance | and the left hip. |
| 3 | Right Elbow Lateral | The distance between the right elbow |
| | Distance | and the right hip |
| 4 | Left Arm Side Raise | The distance between the left wrist |
| | | and the left hip. |
| 5 | Right arm lateral raise | The distance between the right wrist |
| | | and the left hip. |
| 6 | Elbow Distance | The distance between two elbows. |
| 7 | Wrist Distance | The distance between the two wrists. |
| 8 | Left Leg Curl | Distance between left hip and left |
| | | ankle. |
| 9 | Right Leg Curl | Distance between right hip and right |
| | | ankle. |
| 10 | Knee Distance | The distance between two knees. |
| 11 | Ankle Distance | The distance between two ankles. |
| 12 | Left Abs Crunches | The distance between the left shoulder |
| | | and the left knee. |
| 13 | Right Abs Crunches | The distance between the right shoulder |
| | | and the right knee. |
| 14 | Left Elbow Knee | The distance between the left elbow and |
| | | the left knee. |
| 15 | Right Elbow Knee | The distance between the right elbow and |
| | | the right knee. |
| 16 | Left Wrist Knee | The distance between the left wrist and |
| | | the left knee. |
| 17 | Right Wrist Knee | The distance between the right wrist and |
| | | the right knee. |

Note that the number of vectors can be changed to suit the training style or sport the user is using.

The machine learning gesture classifier 1109 classifies the user's gestures by using the K-nearest neighbor algorithm based on the feature vectors calculated from the human body markers output from the gesture detection model unit 1108. The machine learning gesture classifier 1109 also calculates the number of repetitions that the user needs to continue to perform training by observing the probability of the user training. For example, if you need to count the number of repetitions a user does a bicep curl, the algorithm looks at the probability of a bicep curl (up) and a bicep curl (down). If the probability of biceps curling (downward) exceeds 80%, the algorithm marks the biceps curling as starting. When the probability of a biceps curl (up) exceeds 80% and the biceps curl is marked as started, the algorithm should mark it as done and increment the repetition counter.

(10) The Third Output Unit 1110

The third output unit 1110 displays the number of repetitions of the exercise program completed by the user. The number of repetitions of the exercise program performed by the user is a positive integer, such as 8 times.

FIG. 11B shows a schematic block diagram of one embodiment of the first deep neural network unit 1103 of the control apparatus of FIG. 11A. As shown in FIG. 11B, the first deep neural network unit 1103 receives data and/or signals input from the signal preprocessing unit 1102 and/or the signal input unit 1101, and provides the first output 1104 with information about the user's performance and an estimated number of remaining repetitions of the movement. The data and/or signals input by the signal input unit 1101 may be formed as a user information vector 1121, which may be directly fed into the feedforward network 1125. Also, data and/or signals input by the signal preprocessing unit 1102 may be input to, for example, a continuous wavelet transform (CWT) layer 1122 to form a wavelet spectrum 1123. The wavelet spectrum 1123 is input to a deep convolutional neural network (DCNN) 1124, for example. The DCNN 1124 may be, for example, the current topmost layer of EfficientNet V2B0. The output of the DCNN 1124 may be input to the feedforward network 1125 through, for example, an embedding process. The feedforward network 1125 performs fatigue analysis on the user's movements through the AI algorithm developed by the present invention, based on the user information vector 1121 and the output of the embedded DCNN 1124, to estimate the remaining number of repetitions the user can do, and output through the first output unit 1104.

Similarly, the second deep neural network unit 1105 can also be formed by, for example, a deep convolutional neural network, respectively, using the AI algorithm of the present invention to suggest an optimal training load to the user according to the current muscle activation signal and the estimated number of remaining repetitions, and the user's optimal training load is given through the second output unit 1106.

In addition, the machine learning posture classifier 1109 uses the AI algorithm of the present invention to calculate an 18-dimensional vector (feature vector) from the human body markers output from the posture detection model unit 1108 to represent the user's ongoing exercise program and exercise state.

One of the main functions of the machine learning based control device of the present invention is to identify muscle fatigue and thereby suggest optimal training loads for the user. The output of the fatigue analysis is real-time identification of muscle fatigue during resistance training, providing scientific evidence for recommending optimal training loads to the user in the next step.

The personal training system of the present invention may also include a database of user and sensor signals. The database can be contained in the control device or as a separate database, e.g., in a database server.

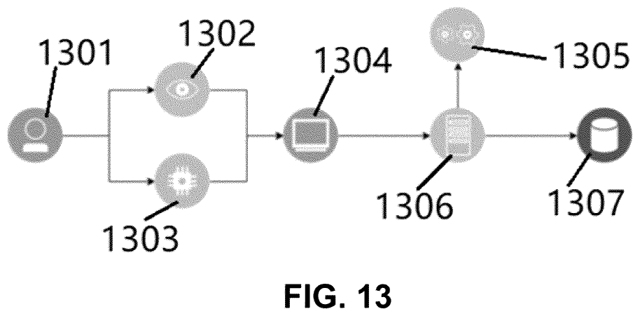

The Inventors have developed a centralized database with various uses, see FIG. 13. FIG. 13 shows a personal training system formed by combining a designated personal training device with the AI algorithm of the control device. As shown in FIG. 13, the personal training system of the present invention may be provided for use by a user 1301, which may include: a camera 1302, a sensor 1303, training software 1304, a machine learning server 1305, an application server 1306, and a database server 1307. According to the example of FIG. 13, the data of user 1301 is captured by camera 1302 (or camera) and sEMG sensor 1303. The sEMG sensor 1303 may correspond to the aforementioned sensor 5 in the user training device. The training software 1304 is responsible for acting as a bridge between the user's data and the application server, i.e. sending the user's data to the application server 1306, and presenting the processing results received from the application server. The application server 1306 is responsible for executing the business logic. For example, user data is received from training software 1304, then sent to machine learning server 1305, and the information received from the machine learning server is routed to the training software, including suggested optimal training loads. The application server 1306 is also responsible for storing user data in the database server 1307, or querying data from the database server. The machine learning server 1305 is responsible for hosting the machine learning algorithms and neural networks, it accepts input from the application server and sends the output to the application server, it hosts the first deep neural network for calculating the remaining number of training iterations and for optimizing the training load The second deep neural network, such as the first deep neural network unit 1103 and the second deep neural network unit 1105 in FIG. 11. The database server 1307 is the persistent layer of data, its role is to persist the data and prepare it to execute data queries received from the application server.

The training software 1304 may be an application program corresponding to the AI fitness training system 1111 of FIG. 11A, which may be downloaded or installed in a user's portable communication device, such as a laptop computer, tablet computer or mobile phone. The database in the database server 1307 may: store collected data; provide data to AI algorithms; and support data analysis. All data collected, used and processed are stored in a database. The data can include, for example, user credentials, training records, sEMG signal readings, and some personalization parameters optimized by AI algorithms.

In addition, the control device of the personal training system of the present invention can also have a coaching module, which can have a GUI and can provide audio feedback, the GUI of the coaching module can be seen in FIG. 14A. The GUI of the coaching module can display training information and real-time visual feedback, a collection of tutorial videos for all exercise/sport types in the exercise library, and provide audio guidance and feedback in real-time.

The GUI allows the user to view all necessary information when performing a resistance training exercise using the personal training device of the present invention. For example, the information may include: (1) current date and time, total training time; (2) workout name included in the current exercise routine, including sets and repetitions, suggested training load; (3) real-time muscle activation level; (4) sensor status; (5) live camera viewer; (6) tutorial video.

The user's real-time muscle activation levels and sensor conditions during exercise can be displayed on a color-coded muscle map. If the control device detects any abnormal condition, a warning will be displayed through the GUI. If a camera is connected to the control, the user can also use the viewer window to check their posture and form.

FIG. 14A shows an example of a schematic design of the GUI of the coaching module. FIG. 14A shows that the user is performing Bent-over Biceps curl 10 times×3 sets of exercise, and the left side of FIG. 14A shows the reference video of the exercise and the weight of the dumbbells used by the user respectively from top to bottom is 10 kilograms (kg), the number of times and sets of the ongoing exercise (the example in FIG. 14A is the second set of ten), and a reference video for the coaching of the exercise, with a schematic diagram showing the name of the exercise the user is doing (bending lumbar biceps curl) and cues for the next exercise (supine triceps stretch), contours of the body and muscles, the location of the various sensors (similar to FIGS. 10A and 10B), and the number of times the current exercise is (The example in FIG. 14A is the 5th time), the date and time are displayed on the right (the example in FIG. 14A is 13:25 on Sep. 1, 2022), and the exercise time (the example in FIG. 14A is that it has exercised for 5 minutes and 36 seconds) and optional/selected exercise (the example in FIG. 14A is the highlighted bicep curl).

FIG. 14B is a schematic diagram of a user wearing the personal training device of the present invention exercising and a corresponding diagram of sensors on the user in the GUI of FIG. 14A. In FIG. 14B, on the left is the user wearing the personal training device of the present invention and is performing a bent over biceps curl (see FIG. 12D), on the right is the sensor in the personal training device worn by the user. In the schematic diagram at the corresponding position on the schematic diagram of the human body, the corresponding icon of each sensor may move correspondingly on the schematic diagram of the human body along with the user's movement.

The Inventors have spent a lot of time in developing the above-mentioned software for the sensor-equipped personal training device and the AI algorithm of the control device. Therefore, the contributing features of the personal training device of the present invention include: the structural design of the sensor pocket to ensure stable skin contact; the optimization of the position where the sensor is inserted into the pocket; the ability of optimizing separately; making samples of various styles, and conducting wearing tests on each subject to find out design deficiencies and wearing difficulties; grading patterns suitable for men's clothing and women's clothing.

The development of AI algorithm software includes: designing and developing AI algorithms to provide more accurate biofeedback and prediction, collecting data for each subject on a six-week fitness training program, training neural networks, calculating K-means The centroids for each exercise of the clustering algorithm, integrating the neural network into the training software system.

As mentioned above, the essential or core features of the present invention include: (1) AI algorithms, including motion recognition algorithms, motion anomaly detection algorithms, and training load recommendation algorithms; (2) personal training devices with wireless sensor interfaces, including sensor pockets and opening design and optimized sensor position. The personal training system of the present invention can solve the following problems: provide a scientific and objective measure of muscle fatigue, rather than a subjective score, during the user's exercise; evaluate exercise form and muscle utilization patterns; minimize risk; and measure and display muscle activation levels during exercise in a user-friendly way.

Therefore, the personal training system of the present invention is capable of measuring and evaluating the muscle activity of the user's major muscle groups, which is not available in other current wearable technologies. The personal training device of the present invention includes garments that allow for easy installation of sensors, which is critical for ordinary users who are less familiar with muscle anatomy. The present invention can be configured to evaluate some of the most common resistance training exercises requiring only dumbbells, without the need for large exercise equipment. The present invention provides users with reliable and real-time feedback on their exercise performance, especially correctness of form and movement, muscle utilization patterns, which are not available in any prior art systems on the market. For example, the corresponding relationship between the contributing technical features of the present invention and the corresponding technical advantages is shown in Table 4 below.

TABLE 4
Technical features of the present invention
and corresponding technical effects.

| Features of the Personal | |
|---|---|
| Training Device | Advantages Derived from |
| of the Invention | the Features |
| Using sEMG technology; an algorithm | Measure muscle activity in a |
| configured to calibrate sensor data. | user-friendly way |
| Measure muscle activity in a user- | |
| friendly way | |
| Tight-fitting garment design, pocket | Simple sensor handling and |
| design. | connection |
| Proven AI algorithms are developed | Reliable evaluation and real-time |
| based on a large amount of reliable | feedback |
| experimental data. | |

By collecting more experimental data to expand the training data set of the AI algorithm, the personal training device of the present invention can further improve the accuracy of the prediction result, verify the prediction result of the AI algorithm through experiments, and further develop the algorithm in the control device, includes motion recognition algorithms and muscle fatigue detection algorithms, visual and auditory feedback in the coaching module.

For example, an application scenario of the personal training system of the present invention may include an AI-assisted training device for resistance training. Moreover, the system architecture and characteristics of the clothing design of the personal training device of the present invention can be applied to other smart wearable technologies, smart wearable devices of advanced or professional training devices requiring real-time biofeedback, and other textile products.

While the present invention has been described in detail in connection with limited embodiments, it is to be understood that the invention is not limited to these disclosed embodiments. Those of ordinary skill in the art can devise other embodiments that are within the spirit and scope of the present invention, including variations, modifications, substitutions, or equivalent arrangements of parts, all of which fall within the scope of the present invention.

What is claimed is:

1. A personal training device comprising:
an upper garment part and a trousers part both for wearing by a user; and
plural sensor accommodating units distributed on the upper garment part and trousers part, plural sensors being installed in the sensor accommodating units such that an individual sensor accommodating unit is equipped with one or more of the sensors, wherein positions of the sensor accommodating units on the upper garment part and trousers part respectively correspond to locations of muscles of major muscle groups of the user such that the sensors, or electrodes thereof, of the sensor accommodating units are positioned on the corresponding muscle locations of the user when the user wears the personal training device, thereby allowing a posture of the user and a muscle activity of the main muscle groups to be monitored during the user doing an exercise.

2. The personal training device of claim 1, wherein the upper garment part and trousers part are separate garment articles, are collectively formed as a one-piece garment, or are formed from plural straps, and wherein the upper garment part and trousers part are tight-fitting or skin-tight.

3. The personal training device of claim 1, wherein the upper garment part, trousers part and sensor accommodating units are made of one or more fabrics, wherein a main fabric selected among the one or more fabrics and used for forming the upper garment part and trousers part is tricot knitted, and wherein a main fabric selected among the one or more fabrics and used for forming the sensor accommodating units is warp-knitted stretch mesh.

4. The personal training device of claim 1, wherein the sensors include surface electromyography sensors and inertial measurement unit sensors, and wherein the muscles of the major muscle groups include upper trapezius, triceps, erector spinae, biceps femoris, pectoralis major, biceps, rectus abdominis, and rectus femoris.

5. The personal training device of claim 1, wherein the sensor accommodating units include 14 units for accommodating 16 sensors.

6. The personal training device of claim 1, wherein the individual sensor accommodating unit is realized as a pocket sewn, snap-attached, or affixed, to the upper garment part or the trousers part.

7. The personal training device of claim 6, wherein the pocket is a Type-1 pocket for accommodating a single sensor, the Type-1 pocket having a length and a width given by L1=(a+c)×p and W1=(b+2c)×p′ where:
L1 is the length of the Type-1 pocket;
W1 is the width of the Type-1 pocket;
a, b and c are length, width and depth of the accommodated sensor, respectively;
p is between 80% and 90% inclusively; and
p′ is between 60% and 65% inclusively.

8. The personal training device of claim 7, wherein the Type-1 pocket includes an inner layer opening, wherein the inner layer opening is rectangular, oval or square in shape, or conforms to a shape of the accommodated sensor, and wherein the inner layer opening has a length and a width given by L3=d+s and W3=e×3 where:
L3 is the length of the inner layer opening;
W3 is the width of the inner layer opening;
d is a sum of lengths of all electrodes in the accommodated sensor;
s is between 2 mm and 4 mm inclusively; and
e is a common width of the electrodes.

9. The personal training device of claim 6, wherein the pocket is a Type-2 pocket for accommodating two sensors, the Type-2 pocket having a length and a width given by L2=(a+c)×p and W2=[(2b+2c)+q]×p′ where:
L2 is the length of the Type-2 pocket;
W2 is the width of the Type-2 pocket;
a, b and c are length, width and depth of an individual accommodated sensor, respectively;
p is between 80% and 90% inclusively;
p′ is between 60% and 65% inclusively; and
q is between 5 mm to 10 mm inclusively.

10. The personal training device of claim 9, wherein the Type-2 pocket includes an inner layer opening, wherein the inner layer opening is rectangular, oval or square in shape, or conforms to a shape of the individual accommodated sensor, and wherein the inner layer opening has a length and a width given by L4=d+s and W4=e×3+f×2 where:
L4 is the length of the inner layer opening;
W4 is the width of the inner layer opening;
d is a sum of lengths of all electrodes in the individual accommodated sensor;
s is between 2 mm and 4 mm inclusively; and
e is a common width of the electrodes.

11. The personal training device of claim 6 further comprising a wire opening located on an outside part of the pocket and spaced from a sensor insertion opening of the pocket by 1 cm to 2 cm, wherein the wire opening is parallel to a side edge of the pocket or angled to a side of the pocket to facilitate placement of sensor electrodes through the wire opening.

12. A control device for processing data generated by a personal training device comprising:
an input unit for receiving detected-signal data from sensors on the personal training device and inputting a user profile of a user of the personal training device;
a database for storing the received detected-signal data and the user profile;
a data preprocessing unit for cleaning and preprocessing the detected-signal data;
a training analysis unit for executing an artificial intelligence algorithm to perform fatigue analysis on the user's movement and providing training load recommendations based on the user profile and the detected-signal data; and
an output unit configured to output an estimated remaining number of repetitions of an exercise to be performed by the user, a training load suggestion given by the training analysis unit, or a number of repetitions of the exercise completed by the user.

13. The control device according to claim 12 further comprising a coaching module having a graphical user interface, the graphical user interface being used for displaying training information and real-time visual feedback of the user's movement, tutorial videos for different types of exercise as stored in the device's exercise library, as well as used for providing audio guidance and feedback on the exercise in real time.

14. The control device according to claim 12, wherein the training analysis unit comprises a first deep neural network unit and a second deep neural network unit, wherein the first neural network unit is configured to estimate the user movement according to a current muscle activation signal, and wherein the second deep neural network unit is configured to suggest an optimal training load to the user according to the current muscle activation signal and the estimated remaining number of repetitions.

15. The control device according to claim 12 further comprising a posture detection model unit and a machine learning posture classifier, wherein the posture detection model unit is configured to receive a signal of the user's human body detected from a camera input unit, and to generate a human body marker based on the detected signal of the user's human body, and wherein the machine learning gesture classifier is configured to calculate a vector representing the user's ongoing exercise program and a motion state from the human body marker generated by the posture detection model unit, whereby the control device is realized as an artificial intelligence fitness training system.

16. The control device according to claim 12, wherein the data preprocessing unit includes a signal preprocessing unit for performing one or more of the following preprocessing functions on the detected-signal data: bandpass filtering; highpass filtering; lowpass filtering; root mean square calculation; moving average calculation; mean absolute value calculation; median frequency calculation; data segmentation; data normalization; and data anomaly identification.

17. A personal training system comprising a personal training device and a control device, wherein:
the personal training device comprises:
an upper garment part and a trousers part both for wearing by a user; and
plural sensor accommodating units distributed on the upper garment part and trousers part, plural sensors being installed in the sensor accommodating units such that an individual sensor accommodating unit is equipped with one or more of the sensors, wherein positions of the sensor accommodating units on the upper garment part and trousers part respectively correspond to locations of muscles of major muscle groups of the user such that the sensors, or electrodes thereof, of the sensor accommodating units are positioned on the corresponding muscle locations of the user when the user wears the personal training device, thereby allowing a posture of the user and a muscle activity of the main muscle groups to be monitored during the user doing an exercise;
and
the control device comprises:
an input unit for receiving detected-signal data from sensors on the personal training device and inputting a user profile of a user of the personal training device;
a database for storing the received detected-signal data and the user profile;
a data preprocessing unit for cleaning and preprocessing the detected-signal data;
a training analysis unit for executing an artificial intelligence algorithm to perform fatigue analysis on the user's movement and providing training load recommendations based on the user profile and the detected-signal data; and
an output unit configured to output an estimated remaining number of repetitions of an exercise to be performed by the user, a training load suggestion given by the training analysis unit, or a number of repetitions of the exercise completed by the user.

18. The personal training system of claim 17 further comprising:
a machine learning server for executing artificial intelligence algorithms for machine learning;
an application server for storing one or more application programs of the control device, allowing the user to download the one or more application programs; and
a database server for storing collected data, providing data to the artificial intelligence algorithm and supporting data analysis.

19. The personal training system of claim 17, wherein the sensors include surface electromyography sensors and inertial measurement unit sensors.

20. The personal training system according to claim 17, wherein the muscles of the major muscle groups include upper trapezius, triceps, erector spinae, biceps femoris, pectoralis major, biceps, rectus abdominis, and rectus femoris.
